(19) United States
(12) Patent Application Publication
(10) Pub. No.: US 2012/0171125 A1
(43) Pub. Date: Jul. 5, 2012

(54) Methods for Enhancing the Cognitive Function

(75) Inventors: Benoît Kenda, Brussels (BE); Alain Matagne, Brussels (BE)

(73) Assignee: UCB PHARMA, S.A., Brussels (BE)

(21) Appl. No.: 13/388,875

(22) PCT Filed: Aug. 4, 2010
(86) PCT No.: PCT/EP2010/004770

§ 371 Date: Mar. 21, 2012

(30) Foreign Application Priority Data
Aug. 7, 2009 (EP) 09100369.9

(51) Int. Cl. A61K 49/00; A61K 31/4178; A61P 25/28; A61P 25/18; A61P 25/24; A61P 25/16; C07D 403/06; A61P 25/00

(52) U.S. Cl. 424/9.2; 548/314.7; 514/397

(57) ABSTRACT

A compound having an affinity to a SV2 protein for the treatment of a condition associated with enhancement or improvement of the cognitive ability or to counteract cognitive decline of a mammal. For example, a compound which is covered by formula (I) R¹ is an halogen atom, preferably, a chlorine or a fluorine atom; n is equal to 1, 2 or 3; and R² is cyano.

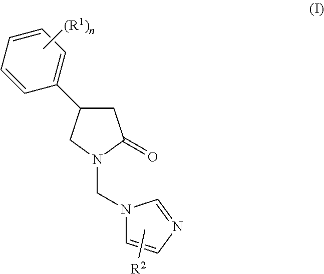

### FIELD OF THE INVENTION

The invention relates to compositions and methods for enhancing the cognitive function or to counteract cognitive decline in a mammal.

### BACKGROUND OF THE INVENTION

Cognitive disorders, i.e. impairments of memory and learning processes, have a significant detrimental effect on the quality of life of patients affected by it. Clinically recognized cognitive disorders vary from mild cognitive impairment through to dementia of varying severity.

Mild cognitive impairment (“MCI”) is believed to be a transition stage between the cognitive changes of normal aging and the more serious problems caused by Alzheimer's disease. Dementia is a clinically recognised broad-spectrum syndrome entailing progressive loss of cognitive capabilities. Dementia can be one of many symptoms of various neurological diseases or the main abnormality associated with the disease, as it is the case in Alzheimer's disease. Most common causes of dementia include: cerebral atrophy associated with Alzheimer's disease, Lewy-bodies disease, front-temporal lobe degeneration, Pick's disease; vascular narrowing or blockage in the brain (i.e. vascular dementia also known as multi-infarct dementia); Huntington's disease, Parkinson's disease; head trauma; HIV infection or Down's syndrome.

Alzheimer's disease (AD) is a progressive degenerative disease of the brain primarily associated with aging. AD is one of several disorders that cause the gradual loss of brain cells and is one of and possibly the leading cause of dementia. Clinical presentation of AD is characterized by loss of memory, cognition, reasoning, judgment, and orientation. Mild cognitive impairment (MCI) is often the first identified stage of AD. As the disease progresses, motor, sensory, and linguistic abilities also are affected until there is global impairment of multiple cognitive functions. These cognitive losses occur gradually, but typically lead to severe impairment, and the disease leads eventually to death in the range of three to twenty years.

Currently there are only a few medications that have been shown to afford at most a modest, mostly transient benefit to the patients suffering from cognitive impairment. Cholinesterase inhibitors (anticholinesterases), such as donepezil (Aricept®), galantamine (Razadyne®), Razadyne ER®, Reminyl®, Nivalin® and rivastigmine tartrate (Exelon®) have been shown to be efficacious in mild to moderate Alzheimer's disease dementia. Exelon® has recently been approved for the treatment of mild to moderate dementia associated with Parkinson's disease. Memantine NMDA receptor antagonists are the first approved Alzheimer's disease medication acting on the glutamatergic system (Axura®, Akatinol®, Namenda®, Ebixa®). These drugs however have not only proven limited efficacy but also considerable side effects which in some cases lead to discontinuation of the therapy. With the increase in the life span and general aging of the population there is a need to develop drugs which could delay or alleviate the cognitive function in aging patients.

The Synaptic Vesicle Protein 2 (SV2) family of synaptic vesicle proteins was first identified with a monoclonal antibody prepared against cholinergic vesicles from the electric organ of the marine ray D. ommata (Buckley et al., J. Cell Biol. 100: 1284-1294 (1985)). Cloning of the individual family members labeled by the antibody resulted in the identification of three different isoforms, SV2A (Bajjalieh et al., Science 257: 1271-1273 (1992)), SV2B (Feany et al., Cell 70(5): 861-867. 1992) and SV2C (Janz and Sudhof, Neuroscience 94(4): 1279-1290 (1999)), all of which react with the original antibody. The overall homology between the three rat isoforms is approximately 60%, with SV2A and SV2C being more similar to each other than SV2B (Janz & Sudhof, Neuroscience 94(4): 1279-1290 (1999)).

The SV2 proteins are integral membrane proteins and have significant but low-level homology (20-30%) to the twelve transmembrane (TM) family of bacterial and fungal transporter proteins that transport sugar, citrate, and xenobiotics (Bajjalieh et al., Science 257: 1271-1273 (1992)). As members of the 12-TM superfamily, SV2 proteins display several unique features. They have relatively short free N- and C-termini and short loops connecting the TM segments. Two notable exceptions, however, are the long cytoplasmic loop between transmembrane regions 6 and 7 and the intra-vesicular loop between transmembrane regions 7 and 8 (which contains 3 N-glycosylation sites).

As a family, SV2 proteins are widely distributed in the brain and in endocrine cells. The three isoforms overlap significantly in their distribution, and can be found co-expressed in the same neuron, and even on the same synaptic vesicle. One isoform or another of the SV2 proteins seems to be present on all synaptic vesicles, and they are probably not limited to neurons that contain any specific neurotransmitters, although one study reports that cholinergic vesicles may not contain SV2 (Blumberg et al., J. Neurochem. 58(3): 801-810 (1992)). SV2 proteins are therefore one of the most common proteins of synaptic vesicles, and have been implicated in the control of calcium-mediated exocytosis of synaptic vesicles. SV2 proteins have also been shown to be expressed in endocrine cells and, along with the additional synaptic vesicle membrane integral proteins p38 and p65, has been demonstrated to be present in endocrine dense core granule membranes (Lowe et al., J. Cell. Biol. 106(1): 51-59 (1988)). SV2A, the most common SV2 isoform, is expressed ubiquitously throughout the brain, and is present as well in secretory granules of endocrine cells. SV2B, while broadly distributed in the brain, is undetected in several brain structures, including the dentate gyrus of the hippocampus, the globus pallidus, reticular nuclei of the thalamus, and the reticular part of the substantia nigra (Bajjalieh et al., 1994). By contrast, SV2C has quite a limited distribution and is found primarily in the phylogenetically old regions such as the pallidum, the substantia nigra, the midbrain, the brainstem and the olfactory bulb. It is undetectable in the cerebral cortex and the hippocampus, and found at low levels in the cerebellar cortex (Janz and Sudhof, Neuroscience 94(4): 1279-1290 (1999)).

In addition to the SV2 protein, the synapse contains other unique regulatory proteins such as synapsin, synaptotagmin and CAPS, which may mediate vesicle fusion or budding. SV2A may be a Ca²⁺ regulatory protein essential for the formation of pre-fusion complexes called SNARE complexes (Xu et al. Cell 99(7): 713-722 (1999)), which include the synaptic vesicle-associated VAMP/synaptobrevin and the plasma membrane proteins syntaxin and SNAP-25. Upon Ca²⁺ accumulation in the synapse the binding of synaptotagmin to SV2A is inhibited and the dimerization of two synaptotagmin Ca²⁺ binding domains is stimulated (Bajjalieh, Curr. Opin. Neurobiol. 9(3): 321-328 (1999)). This dimerization may play a role in organizing the SNARE complex and promoting vesicle fusion, as at low Ca²⁺ concentrations, SV2A remains bound to synaptotagmin and fusion will not occur.

The affinity of SV2A for synaptotagmin is regulated by the phosphorylation of the amino terminus of SV2 (Pyle et al., J. Biol. Chem. 275(22): 17195-17200 (2000)). The possibility that SV2 proteins play a role in either Ca²⁺ transport, or regulation in the synaptic vesicle has been supported by studies of SV2A and SV2B knockout animals (Janz et al., Neuron 24: 1003-1016 (1999)). An alternative hypothesis is that the SV2 proteins, while derived from transport proteins, now serve a different function in the vesicle, whether a structural role or a role in regulation of vesicle fusion or recycling and the exocytotic release of their contents (Janz and Sudhof, Neuroscience 94(4): 1279-1290 (1999)).

There have been two reports of SV2 protein knockout mice: one that examines only SV2A knockouts (Crowder et al., Proc. Nat. Acad. Sci. USA 96(26): 15268-15273 (1999)) and the other which looks at both SV2A and SV2B knockout animals, as well as the SV2A/SV2B double knockout (Janz et al., Neuron 24: 1003-1016 (1999)).

Animals homozygous for SV2A gene disruption appear normal at birth, but fail to grow, experience severe seizures, and die within the first few weeks postnatal. SV2A homozygous knockout mice experience seizures that are longer lasting, stronger, and more debilitating than any other mouse strain (Janz et al., Neuron 24: 1003-1016 (1999)). Despite the appearance of postnatal seizures, all SV2A knockout animals have completely normal gross brain morphology, including normal levels of the tested synaptic proteins. Furthermore, the hippocampal neuronal cultures from both SV2A and SV2A/SV2B double knockout mice formed synapses that were ultrastructurally normal, and had unchanged size, number and location of synaptic vesicles (Janz et al., Neuron 24: 1003-1016 (1999); Crowder et al., Proc. Nat. Acad. Sci. USA 96(26): 15268-15273 (1999)). Unlike the frequently observed seizures caused by structural and developmental abnormalities easily detected in many other type of knockouts, the SV2A knockout mice show a strong seizure phenotype with no associated macro or micro scale abnormalities of the brain or synapse. As another marker of brain function, studies of synaptic transmission in primary neuronal cultures from SV2A, SV2B, and SV2A/SV2B knockout mice indicate that the sizes and frequencies of sIPSCs and of spontaneous excitatory postsynaptic currents (sEPSCs), are normal. Electrical stimulation induced robust EPSCs and IPSCs in the cultured neurons from all genotypes.

In contrast to SV2A, SV2B knockout mice reveal no overt pathology (Janz et al., 1999). It is believed that one possible reason for this lack of consequence of loss of SV2B is that can be functionally replaced by SV2A, which appears to be co-expressed everywhere SV2B is normally expressed.

While the function of SV2A and other family members still remains unknown, one hypothesis is that this transporter homologue is a functional transporter for some common synaptic vesicle molecule. More specifically, there is evidence linking SV2A to the regulation of calcium-mediated vesicle exocytosis, and as a result, it is thought that it may be a Ca²⁺ transporter. SV2A and other family members may also have roles in the function of synaptic vesicles. Such roles may include modulating aspects of their formation, loading with neurotransmitter, fusion with the plasma membrane, re-cycling, and interactions with other proteins and cellular compartments and organelles. For instance it has been shown that SV2 proteins can interact with the synaptic vesicle protein synaptotagmin and the extracellular matrix protein laminin-1 (Carlson, Perspect. Dev. Neurobiol. 3(4): 373-386 (1996)). The SV2 proteins may play important roles in regulating cytoplasmic or organellar calcium levels at the presynaptic terminal, and may also interact with N-type calcium channels on the plasma membrane, either directly or indirectly.

Levetiracetam or (S)-(−)-alpha-ethyl-2-oxo-1-pyrrolidine acetamide, is a laevorotatory compound, disclosed in the European patent No. EP 0 162 036 B as being a protective agent for the treatment and the prevention of hypoxic and ischemic type aggressions of the central nervous system. Levetiracetam has the following structure:

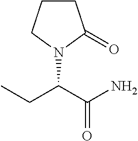

Levetiracetam has been approved, and is marketed as Keppra®, in many countries including the European Union and the United States for the treatment of various forms of epilepsy, a therapeutic indication for which it has been demonstrated that its dextrorotatory enantiomer (R)-(+)-alpha-ethyl-2-oxo-1-pyrrolidine acetamide completely lacks activity (Gower et al., Eur. J. Pharmacol. 222: 193-203 (1992)).

In 2004, it was shown that SV2A is the brain binding site of levetiracetam (Lynch et al., Proc. Natl. Acad. Sci. 101: 9861-9866 (2004)), but the molecular mechanism underlying the therapeutic action of levetiracetam is not yet fully understood. More recently it was suggested that SV2-mediated vesicle priming could be regulated by adenine nucleotides, which might provide a link between cellular energy levels and regulated secretion (Yao and Bajjalieh, J. Biol. Chem. 283(30): 20628-20634 (2008)).

It has been repeatedly reported however that levetiracetam has no impact on the cognitive function both in animals as well as in humans (Lamberty et al, Epilepsy & Behavior 1, 333-342 (2000); Klitgaard et al. Epilepsy Research 50, 55-65 (2002); Shannon H & Love, P. Epilepsy & Behavior 7, 620-628 (2005); Higgins et al. Psychopharmacology 207, 513-527 (2010)).

Further racetam-type drugs include piracetam, oxiracetam, aniracetam, pramiracetam and phenylpiracetam, which have been used in humans and some of which are available as dietary supplements. Of these, oxiracetam and aniracetam are no longer in clinical use. Pramiracetam reportedly improved cognitive deficits associated with traumatic brain injuries. Although piracetam exhibited no long-term benefits for the treatment of mild cognitive impairments, recent studies demonstrated its neuroprotective effect when used during coronary bypass surgery. It was also effective in the treatment of cognitive disorders of cerebrovascular and traumatic origins; however, its overall effect on lowering depression and anxiety was higher than improving memory. As add-on therapy, it appears to benefit individuals with myoclonus epilepsy and tardive dyskinesia. Phenylpiracetam is more potent than piracetam and is used for a wider range of indications. In combination with a vasodilator drug, piracetam—which does not bind to SV2A—appeared to have an additive beneficial effect on various cognitive disabilities.

Seletracetam ((2S)-2-[(4S)-4-(2,2-difluorovinyl)-2-oxo-pyrrolidinyl]butanamide) and brivaracetam ((2S)-2-[(4R)-2-oxo-4-propyl-pyrrolidin-1-yl]butanamide) show both potent affinity to SV2A, very potent antiepileptic activities and are there in clinical development, however no meaningful activity on the cognitive function was reported so far.

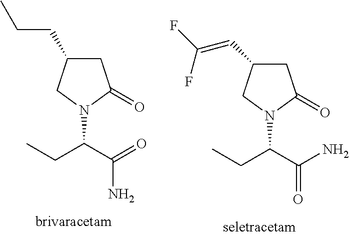

Both compounds have been first disclosed in WO 01/62726.

### SUMMARY OF THE INVENTION

The present invention relates to compounds, compositions and methods for the treatment of conditions associated with enhancement or improvement of cognitive ability or to counteract cognitive decline. More particularly, the invention relates to methods for enhancing cognitive function by administering compounds that have the two following properties, i.e.
They have an affinity to a SV2 protein; and
They counteract the antiepileptic or anti-convulsive activity of levetiracetam or of brivaracetam or of seletracetam, e.g. in animal models mimicking epilepsy, thereby acting as functional antagonists.

Thus, the affinity of the candidate compound for a SV2 protein is a necessary condition but said affinity alone is not sufficient for the compound to be suitable for the treatment of conditions associated with enhancement or improvement of cognitive ability or to counteract cognitive decline; said candidate compound must furthermore counteract, down-regulate or off-set the primary therapeutic effect of either of the anti-convulsants which are levetiracetam or brivaracetam or seletracetam.

In a specific embodiment, said compounds have an affinity to the SV2A protein.

In a further specific embodiment, said compounds have an affinity to the SV2B protein.

In a further specific embodiment, said compounds have an affinity to the SV2C protein.

In a specific embodiment, said compounds counteract the antiepileptic or anti-convulsive activity of levetiracetam, e.g. in animal models mimicking epilepsy set out below.

A further aspect of the present invention consists in the identification of compounds useful in treatment of conditions associated with enhancement or improvement of cognitive ability or to counteract cognitive decline by
Determining whether a test compound has a SV2 binding affinity; and
Determining whether a test compound counteracts the antiepileptic or anti-convulsive activity of levetiracetam.

A further aspect of the present invention consists in pharmaceutical compositions containing a compound which has been identified pursuant to the above set out method and which may furthermore contain a pharmaceutically acceptable excipient.

Further aspects of the invention will become apparent from the detailed specification.

### DETAILED DESCRIPTION OF THE INVENTION

The present invention is based on the surprising finding that compounds having on the one hand an affinity to a SV2 protein and which on the other hand counteract the well-known anti-epileptic activity of levetiracetam may be used in enhancing the cognitive function of a mammal in need of, in particular of a human.

“Affinity” used throughout this specification shall mean that the molecules according to the present invention are binding to a SV2 protein, e.g. SV2A protein, or a SV2B protein or a SV2C protein. Binding assays involving a SV2 protein are known to a person skilled in art (e.g. from WO 2005/054188). In one embodiment the binding affinity of the molecules of the present invention display a pIC₅₀ value of at least 5, more preferably at least 6, more preferably at least 7 and most preferably at least 8.

In one embodiment the molecules according to the present invention bind at least to SV2A. In a further embodiment the molecules according to the present invention bind to SV2A and SV2C. In still a further embodiment, the molecules according to the present invention bind to SV2A and SV2B. In still a further embodiment, the molecules according to the present invention bind to SV2B and SV2C. In still a further embodiment, the molecules according to the present invention bind to SV2A and SV2B and SV2C.

In still a further embodiment, the molecules according to the present invention bind selectively to SV2B.

In still a further embodiment, the molecules according to the present invention bind selectively to SV2C.

In still a further embodiment, the molecules according to the present invention have neuroprotective properties. The term “neuroprotective properties” refers to the capacity of the molecules according to the present invention to influence mechanisms within the nervous system which protect neurons from apoptosis or degeneration, for example following a brain injury or as a result of chronic neurodegenerative diseases.

“To counteract the anti-epileptic activity of levetiracetam or of brivaracetam or of seletracetam” used throughout this specification shall mean that the compounds according to the present invention diminish, or down-regulate or off-set the anti-convulsive and/or anti-epileptic activity of levetiracetam or of brivaracetam or of seletracetam or of any other compound interacting with the levetiracetam binding site on the SV2 protein. The anti-epileptic activity of levetiracetam may be determined according to methods that are known to a person skilled in art, e.g. using the “sound-susceptible mice model” described e.g. in WO 2005/054188, or the amygdala kindling rat model described e.g. in Löscher et al., Exp. Neurol. 93: 211-226 (1986).

In the sound-susceptible mice model, the compounds according to the present invention diminish significantly the antiepileptic activity of levetiracetam by producing a shift of its ED₅₀ value (dose protecting 50% of the animals) against clonic seizures towards higher doses, at least by a factor of 2 or 3. Most preferably, they shift the antiepileptic activity of levetiracetam (brivaracetam/seletracetam) towards higher doses by a factor higher or equal to 5.

Thus, molecules according to the present invention having the above set of properties of binding to a SV2 protein and counteracting the anti-epileptic activity of levetiracetam may be identified using routine assay methods know to a person skilled in the art. Such compounds are believed to be useful in the treatment of a cognitive disorder or for improving the cognitive function or counteracting the decline of the cognitive function.

The terms “treatment of conditions associated with enhancement or improvement of cognitive ability” or “to counteract cognitive decline” or “treatment of a cognitive disorder” or “improving the cognitive function” or “counteracting the decline of the cognitive function” used throughout this specification shall mean promoting cognitive function (affecting impaired cognitive function in the subject so that it more closely resembles the function of an aged-matched normal, unimpaired subject, including affecting states in which cognitive function is reduced compared to a normal subject) and preserving cognitive function (affecting normal or impaired cognitive function such that it does not decline or does not fall below that observed in the subject upon first presentation or diagnosis, e.g. to the extent of expected decline in the absence of treatment). The suitability of the compounds according to the present invention for conditions associated with enhancement or improvement of cognitive ability may be tested through assays that are well known in the art. Such assays include in particular the novel object recognition test (NOR) set out in Example 5 as well as the Y-maze test set out in Example 6.

In one embodiment of the invention, the mammal has normal cognitive function which is improved.

In a further embodiment the mammal exhibits cognitive impairment associated with aging.

In still a further embodiment the mammal is a human with cognitive impairment associated with a disease or disorder such as autism, dyslexia, attention deficit hyperactivity disorder, schizophrenia, obsessive compulsive disorders, psychosis, bipolar disorders, depression, Tourette's syndrome and disorders of learning in children, adolescents and adults, Age Associated Memory Impairment, Age Associated Cognitive Decline, Parkinson's Disease, Down's Syndrome, traumatic brain injury Huntington's Disease, Progressive Supranuclear Palsy (PSP), HIV, stroke, vascular diseases, Pick's or Creutzfeldt-Jacob diseases, multiple sclerosis (MS), other white matter disorders and drug-induced cognitive worsening.

In still a further embodiment, the impairment of cognitive function is caused by, or attributed to, Alzheimer's disease. In another embodiment, the impairment of cognitive function is caused by, or attributed to, mild cognitive impairment (MCI).

The compounds according to the present invention may be used for the manufacture of a pharmaceutical composition for the treatment of a cognitive disorder or for improving the cognitive function or counteracting the decline of the cognitive function. Such compositions typically contain the active pharmaceutical ingredient and a pharmaceutically acceptable excipient.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diaminetetraacetic acid, buffers such as acetates, citrates or phosphates and agents for adjusting the osmolarity, such as sodium chloride or dextrose.

Also comprised by the present invention are pharmaceutical compositions containing the compound of the present invention in the form of a pharmaceutically acceptable co-crystal.

Such pharmaceutical compositions may furthermore contain known or marketed therapeutic agents used in the treatment of cognitive or a neurological disorders (AD) including donepezil (Aricept®), galantamine (Razadyne®), Razadyne ER®, Reminyl®, Nivalin®, rivastigmine tartrate (Exelon®), Exelon® (Axura®, Akatinol®, Namenda®, Ebixa®).

In an embodiment the compounds useful for the treatment according to the present invention include those of formula (I)

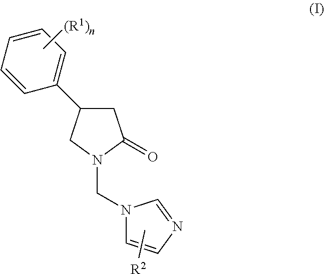

wherein
R¹ is an halogen atom, preferably a chlorine or a fluorine atom;
n is equal to 1, 2 or 3; and
R² is cyano.

Specific compounds of the present invention are those selected from the group consisting of:
(−)-1-{[2-oxo-4-(2,3,4-trifluorophenyl)pyrrolidin-1-yl]methyl}-1H-imidazole-5-carbonitrile;
(+)-1-{[2-oxo-4-(2,3,4-trifluorophenyl)pyrrolidin-1-yl]methyl}-1H-imidazole-5-carbonitrile;
(+)-1-{[2-oxo-4-(3,4,5-trifluorophenyl)pyrrolidin-1-yl]methyl}-1H-imidazole-4-carbonitrile;
(+)-1-{[2-oxo-4-(2,4,5-trifluorophenyl)pyrrolidin-1-yl]methyl}-1H-imidazole-4-carbonitrile;
(−)-1-{[2-oxo-4-(3,4,5-trifluorophenyl)pyrrolidin-1-yl]methyl}-1H-imidazole-5-carbonitrile; and
(+)-1-{[2-oxo-4-(3,4,5-trifluorophenyl)pyrrolidin-1-yl]methyl}-1H-imidazole-5-carbonitrile

Compounds according to formula (I) may be prepared according to methods disclosed in WO 2005/054188.

Further compounds that are useful according to the present invention include:
(+)-1-[(2-aminopyridin-4-yl)methyl]-4-(3,4,5-trifluorophenyl)pyrrolidin-2-one, and
6-methyl-4H-pyrido[1,2-a]pyrimidin-4-one (CAS RN 23443-11-0).

The term “halogen”, as used herein, includes an atom of chlorine, bromine, fluorine, iodine. Preferred halogens are chlorine and fluorine.

The term “cyano”, as used herein, represents a group of formula —CN.

The pro-cognitive activity of the compounds according to the present invention in particular of formula I, or their pharmaceutically acceptable salts, may be determined by a variety of preclinical tests and models known to a skilled person in the art. Such tests may challenge the efficacy on multiple memory phases and types. In contrast to challenging a particular memory types or phases, the cognitive models test the ability of a compound to prevent or reverse a memory deficit in a given brain pathway, system, or function.

In pre-clinical animal models, the compounds according to the present invention improve cholinergic memory deficit induced by scopolamine, a muscarinic receptor antagonists. They also improve the memory deficit induced by beta amyloid. Memory deficits in Alzheimer disease may have both a cholinergic origin as a consequence of specific cholinergic degeneration during disease progression, and an amyloid origin as a consequence of beta amyloid increase in the brain. Therefore, it is believed that the compounds according to the present invention have a strong potential to improve cognitive deficits in Alzheimer disease.

The compounds according to the present invention show a strong efficacy to improve short and long term memory as seen by reversing the scopolamine induced deficit in the novel object recognition and passive avoidance, respectively. They also improve spatial reference learning as seen by improved amyloid-induced memory deficit in the Morris water maze.

In addition, the compounds according to the present invention may also improve attention in the pre-pulse inhibition and the 5-choice test; it may improve social recognition memory, as well as associative memory tested in the active avoidance test.

The compounds according to the present invention show a strong efficacy to improve two phases of memory: acquisition, and consolidation. They improve the acquisition phase of short and long term memory as seen by reversing the scopolamine induced deficit in the novel object recognition and passive avoidance, respectively. They improve the consolidation of spatial reference learning as seen by improved amyloid-induced memory deficit in the Morris water maze. Efficacy to improve retention might also be found in the inhibitory avoidance or active avoidance test.

### EXAMPLES

### Example 1

### Synthesis of 1-[(2-aminopyridin-4-yl)methyl]-4-(3,4,5-trifluorophenyl)-pyrrolidin-2-one and its enantiomers

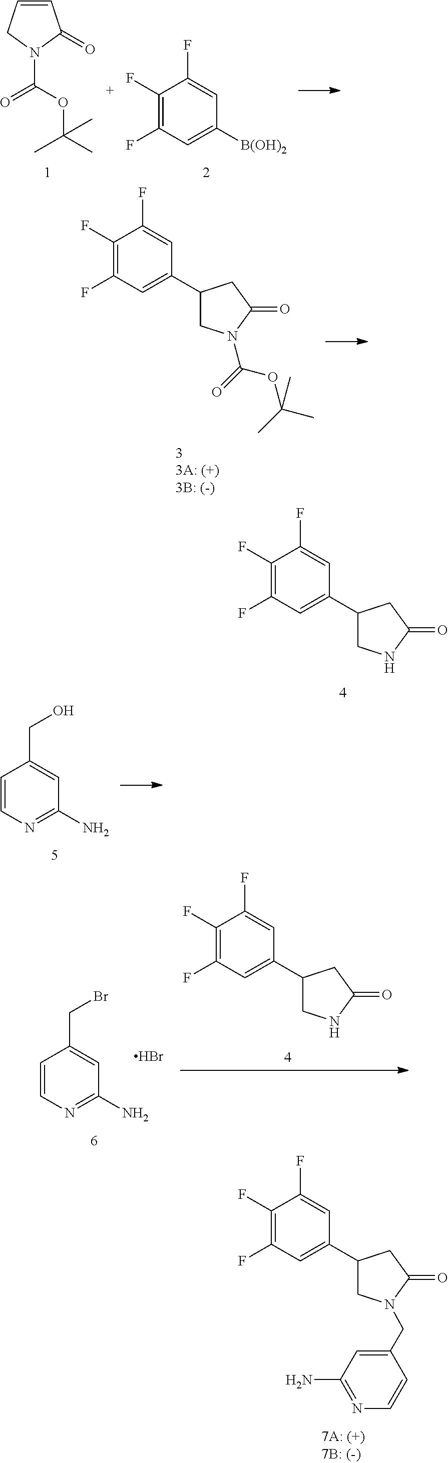

### 1.1 Synthesis of tert-butyl 2-oxo-4-(3,4,5-trifluorophenyl)pyrrolidine-1-carboxylate 3 and enantiomers

To a solution of tert-butyl 2-oxo-2,5-dihydro-1H-pyrrole-1-carboxylate 1 (10 g, 1 eq., 54.6 mmol) in dioxane/water (100 mL/30 mL) are added (3,4,5-trifluorophenyl)boronic acid 2 (19.2 g, 2 eq., 109.2 mmol), cesium fluoride (24.9 g, 3 eq., 163.8 mmol), 2,2′-bis(diphenyl-phosphino)-1,1′-binaphtyl (1.5 g, 4.5%, 2.5 mmol), potassium carbonate (22.6 g, 3 eq., 163.8 mmol) and chloro(1,5-cyclooctadiene)rhodium(I)dimer (0.82 g, 1.5%, 8.2 mmol) at room temperature. The mixture is heated at 110° C. for 2 h. Solvent are removed under reduced pressure and the residue is purified by chromatography over silicagel (CH₂Cl₂/MeOH/NH₄OH 96/3.5/0.5 v/v/v) to afford tert-butyl 2-oxo-4-(3,4,5-trifluorophenyl)pyrrolidine-1-carboxylate 3. The enantiomers are resolved by chiral chromatography (chiralpak IC, 150*4.6 mm, eluent: heptane/AcOEt/DEA 80/20/0.1 v/v/v) to afford (+)-tert-butyl 2-oxo-4-(3,4,5-trifluorophenyl)-pyrrolidine-1-carboxylate 3A (second eluted, 5.1 g), and its enantiomer (−)-tert-butyl 2-oxo-4-(3,4,5-trifluorophenyl)pyrrolidine-1-carboxylate 3B (first eluted, 5.2 g) as white solids.

LC-MS (MH⁺): 316; Compound 3A: alpha_{D} (MeOH, 22° C.): −0.052°

### 1.2 Synthesis of (−)-4-(3,4,5-trifluorophenyl)pyrrolidin-2-one 4

At 0° C., TFA (20 mL, 261 mmol) is added to a solution of (+)-tert-butyl 2-oxo-4-(3,4,5-trifluorophenyl)pyrrolidine-1-carboxylate 3A (8 g, 1 eq., 25.4 mmol) in dichloromethane (100 mL). The mixture is stirred at room temperature for 2 h. Then, TFA and solvent are removed under reduced pressure. The crude mixture is poured in an aqueous saturated solution of NaHCO₃ (100 mL) and extracted with AcOEt (3*200 mL), the combined organic extracts are dried over MgSO₄ and after filtration, concentrated under reduced pressure. The conversion is total and the evaporation affords 5.5 g of (−)-4-(3,4,5-trifluorophenyl)pyrrolidin-2-one 4, which is used in the next step without any further purification.

LC-MS (MH⁺): 216; LC-MS (MH⁻): 214; alpha_{D} (MeOH, 22° C.): −0.227°

### 1.3 Synthesis of 4-(bromomethyl)pyridin-2-amine hydrobromide 6

At 0° C., bromine (0.894 mL, 1.2 eq., 17.4 mmol) is added drop wise to a solution of triphenylphosphine (4.18 g, 1.1 eq., 16 mmol) in acetonitrile (90 mL). After 30 min at room temperature, (2-aminopyridin-4-yl)methanol 5 (1.8 g, 1 eq., 14.5 mmol) is added to the mixture. After overnight stirring at room temperature, the mixture is filtered and the solid is washed twice with ether (100 mL). The organic extracts are combined and concentrated under reduced pressure to afford crude 4-(bromomethyl)pyridin-2-amine hydrobromide 6 which is used for the next step without any further purification.

GC-MS (MH⁺): 186/188.

### 1.4 Synthesis of 1-[(2-aminopyridin-4-yl)methyl]-4-(3,4,5-trifluorophenyl)pyrrolidin-2-one and its enantiomers

NaH (60% in mineral oil, 1.1 g, 3 eq., 27.9 mmol) is added portion wise at 0° C. to a solution of (−)-4-(3,4,5-trifluorophenyl)pyrrolidin-2-one 4 (2 g, 1 eq., 9.3 mmol) in dry DMF (100 mL). After 30 min at room temperature, tetrabutylammonium iodide (343 mg, 0.1 eq., 0.93 mmol) and 4-(bromomethyl)pyridin-2-amine hydrobromide 6 (3 g, 1.2 eq., 11.1 mmol) are added. After overnight stirring, the mixture is quenched with an aqueous saturated NaHCO₃ solution (30 mL) and AcOEt (100 mL). The organic phase is washed with brine (3*300 mL) and the aqueous phase is extracted with AcOEt (2*300 mL). The combined organic extracts are dried over anhydrous MgSO₄ and concentrated under reduced pressure. The crude residue is purified by chromatography on silicagel (CH₂Cl₂/MeOH/NH₄OH 96/3.6/0.4 v/v/v). It is observed by chiral analytical HPLC that an epimerization occurs during this synthesis step. The enantiomers are resolved by chiral chromatography (chiralcel OD-H 250*4.6 mm, eluent: /PrOH/hexane/DEA 50/50/0.1 v/v/v) to afford (−)-1-[(2-aminopyridin-4-yl)methyl]-4-(3,4,5-trifluorophenyl)pyrrolidin-2-one 7A (first eluted, 630 mg) and (+)-1-[(2-aminopyridin-4-yl)methyl]-4-(3,4,5-trifluorophenyl)pyrrolidin-2-one 7B (second eluted, 1.5 g) as white solids.

### Compound 7A:

Yield: 22%; LC-MS (MH+): 322; alpha_{D} (MeOH, 22° C.): −0.495°

¹H NMR (DMSO) δ 7.85 (d, J=5.5 Hz, 1H), 7.32 (m, 2H), 6.39 (m, 1H), 6.35 (s, 1H), 6.10 (s, 2H), 4.36 (m, 1H), 4.19 (m, 1H), 3.63 (m, 2H), 3.24 (m, 1H), 2.72 (m, 1H), 2.57 (M, 1H)

### Compound 7B:

Yield: 52%; LC-MS (MH+): 322; alpha_{D} (MeOH, 22° C.): +0.358°

¹H NMR δ 7.84 (m, 1H), 7.32 (m, 2H), 6.36 (dd, J=5.3, 1.3 Hz, 1H), 6.30 (s, 1H), 5.89 (s, 2H), 4.34 (m, 1H), 4.17 (m, 1H), 3.64 (m, 2H), 3.24 (m, 1H), 2.72 (m, 1H), 2.56 (m, 1H)

### Example 2

### Binding Assay to SV2A

The inhibition constant (Ki) of a compound is determined in competitive binding experiments by measuring the binding of a single concentration of a radioactive ligand at equilibrium with various concentrations of the unlabeled test substance. The concentration of the test substance inhibiting 50% of the specific binding of the radioligand is called the IC₅₀. The equilibrium dissociation constant Ki is proportional to the IC₅₀ and is calculated using the equation of Cheng and Prusoff (Cheng Y. et al., Biochem. Pharmacol. 22: 3099-3108 (1972)).

The concentration range usually encompasses 6 log units with variable steps (0.3 to 0.5 log). Assays are performed in mono- or duplicate, each Kᵢ determination is performed on two different samples of test substance.

Cerebral cortex from 200-250 g male Sprague-Dawley rats are homogenised using a Potter S homogeniser (10 strokes at 1,000 rpm; Braun, Germany) in 20 mmol/l Tris-HCl (pH 7.4), 250 mmol/l sucrose (buffer A); all operations are performed at 4° C. The homogenate is centrifuged at 30,000 g for 15 min. The crude membrane pellet obtained is resuspended in 50 mmol/l Tris-HCl (pH 7.4), (buffer B) and incubated 15 min at 37° C., centrifuged at 30,000 g for 15 min and washed twice with the same buffer. The final pellet is resuspended in buffer A at a protein concentration ranging from 15 to 25 mg/ml and stored in liquid nitrogen.

Membranes (150-200 μg of protein/assay) are incubated at 4° C. for 120 min in 0.5 ml of a 50 mmol/l Tris-HCl buffer (pH 7.4) containing 2 mmol/l MgCl2, 1 to 2 10⁻⁹ mol/l of [3H]-2-[4-(3-azidophenyl)-2-oxo-1-pyrrolidinyl]butanamide and increasing concentrations of the test compound of formula (I). The non specific binding (NSB) is defined as the residual binding observed in the presence of a concentration of reference substance (e.g. 10-3 mol/l levetiracetam) that binds essentially all the receptors. Membrane-bound and free radioligands are separated by rapid filtration through glass fiber filters (equivalent to Whatman GF/C or GF/B; VEL, Belgium) pre-soaked in 0.1% polyethyleneimine and 10-3 mol/l levetiracetam to reduce non specific binding. Samples and filters are rinsed by at least 6 ml of 50 mmol/l Tris-HCl (pH 7.4) buffer. The entire filtration procedure does not exceed 10 seconds per sample. The radioactivity trapped onto the filters is counted by liquid scintillation in a β-counter (Tri-Carb 1900 or TopCount 9206, Camberra Packard, Belgium, or any other equivalent counter). Data analysis is performed by a computerized non linear curve fitting method using a set of equations describing several binding models assuming populations of independent non-interacting receptors, which obey the law of mass.

Compounds of formula (I) according to the invention show pIC₅₀ values with regard to SV2A of at least 5.

### Example 3

### Binding Assay to SV2C

For this assay, SV2C expressed in COS-7 cells are used under standard conditions (see Example 2). [³H]-(+)-4-(3-azido-2,4-difluorophenyl)-1-(1H-imidazol-1-ylmethyl)pyrrolidin-2-one is the used as the radio ligand that binds selectively to SV2C whereby the differential binding of the test compounds is measured, the IC₅₀s of the test compounds are calculated under conditions known to a person skilled in the art.

Test compounds of formula (I) according to the invention showed pIC₅₀ values with regard to SV2C of at least about 5.

### Example 4

### Antagonism of the Protection Afforded by Levetiracetam in the Sound-Susceptible Mice Model of Epilepsy

The objective of testing compounds in an animal model of sound-susceptible mice is to evaluate its ability to reduce the anticonvulsant potency of levetiracetam against clonic convulsions induced in sound-susceptible mice. In this model of primary generalised epilepsy, seizures are evoked without electrical or chemical stimulation and the seizure types are, at least in part, similar in their clinical phenomenology to seizures occurring in man (Löscher W. & Schmidt D., Epilepsy Res. 2: 145-181 (1998); Buchhalter J. R., Epilepsia 34: S31-S41 (1993)).

Male or female genetically sound-sensitive mice (14-28 g; N=10), derived from a DBA strain originally selected by Dr. Lehmann of the Laboratory of Acoustic Physiology (Paris) and bred in Charles River laboratories, France, are used. For testing, the animals are placed in small cages, one mouse per cage, in a sound-attenuated chamber. After a period of orientation of 30 seconds, the acoustic stimulus (90 dB, 10-20 kHz) is delivered for 30 seconds via loudspeakers positioned above each cage. During this interval, the mice are observed and the presence of the 3 phases of the seizure activity namely wild running, clonic and tonic convulsions, is recorded. The proportion of mice protected against clonic convulsions is calculated.

The experimental design consists of several groups, one group receiving the vehicle controls, one group receiving a fixed dose of the test compounds alone and the other groups receiving different doses of levetiracetam alone, or in combination with a fixed dose of the test compounds. The test compounds and levetiracetam are administered intraperitoneally 60 and 30 minutes, respectively, before the induction of audiogenic seizures. The range of the doses administered has a logarithmic progression, generally between 1.0×10⁻⁵ mol/kg and 1.0×10⁻³ mol/kg, but lower or higher doses are tested if necessary.

An ED₅₀ value, i.e. the dose producing 50% protection relative to the control group, together with 95% confidence limits, is calculated on the groups receiving levetiracetam alone and on those receiving levetiracetam together with a fixed dose of the test compounds of formula (I) using a Probit Analysis (SAS/STAT® Software, version 6.09, PROBIT procedure) of the proportion of mice protected against clonic convulsions. A shift in the potency of levetiracetam is then calculated by comparing the respective ED₅₀ values, with and without co-administration of the test compounds.

A shortest version of the experimental design is also used. It consists in four groups of mice, one receiving the vehicle controls, one group receiving levetiracetam alone at a dose providing maximal protection against clonic convulsions (generally 1.8×10⁻⁴ mol/kg), one group received a fixed dose of the test compounds alone (generally 1.0×10⁻⁴ mol/kg but lower or higher doses are tested if necessary) and the last group a combination of levetiracetam and a fixed dose of the test compounds. The proportion of mice protected against clonic convulsions is calculated in each group.

Compounds of formula (I) according to the invention are found to shift towards higher doses the protection afforded by levetiracetam in the sound-susceptible mice model of epilepsy by a factor of at least 2 or 3.

### Example 5

### In Vivo Model for Assessing the Efficacy of a Test Compound in Learning and Memory Disorders (Novel Object Recognition Test; NOR)

Evaluation of promnesiant properties in the mouse model of 2-trial novel object recognition in a situation of scopolamine induced memory deficit: the two-trial object recognition paradigm, initially developed by Ennaceur and Delacour (1988) in the rat, can be considered as a model of episodic-like memory. This learning and memory paradigm is based on spontaneous exploratory activity of rodents and does not involve rule learning or reinforcement. The object recognition paradigm has been shown to be sensitive to the effects of ageing and cholinergic dysfunction (Scali et al, 1994; Bartolini et al, 1996). This model has been adapted to mice and validated using pharmacological agents (Bertaina-Anglade et al, 2003).

The purpose of the study is to evaluate the ability of test compounds to reverse the experimental deficit induced by scopolamine. The experiments was carried out using male C57BL/6J mice (Centre d'Elevage R. Janvier, B. P. 55, 53940 Le Genest-Saint-Isle, F.), weighing 20-35 g (10-14 weeks old) at their arrival that should meet inclusion criteria described in the experimental procedure. The animals were housed in groups of 4-9 in polypropylene cages (floor area=777 cm²) under standard conditions: room temperature (22±2° C.), light/dark cycle (12 h/12 h), water and food (SAFE A04) ad libitum. The experimental arena is a square wooden box (40×40×40 cm) painted in dark blue, with 8*8 cm black painted squares under a clear plexiglass floor. The arena was placed in a dark room illuminated only by lamps giving a uniform dim light in the box (around 60 lux). The day before the test, mice were habituated to the environment for a maximum of 30 min. On experimental day, mice were submitted to two trials spaced by an intertrial interval of 60 min. During the first trial (acquisition trial, T1), mice were placed in the arena containing 2 identical objects and time required by each animal to complete 20 s of object exploration was determined with a cut-off time of 12 min. Exploration was considered to be directing the nose at a distance less than 2 cm from the object and/or touching the object. For the second trial (testing trial, T2), one of the objects presented in the first trial was replaced by an unknown object (novel object), mice were placed back in the arena for 5 min and exploration of each object together with locomotor activity was determined. A criterion of minimal level of object exploration was used in the study to exclude animals with naturally low levels of spontaneous exploration: only animals having a minimal level of object exploration of 3 s during the testing trial (Novel+Familiar≧3 s) were included in the study. The following parameters were measured: time required to achieve 20 s of object exploration on T1 (s), locomotor activity on T1 (number of crossed lines), time spent in active exploration of the familiar object on T2 (s), time spent in active exploration of the novel object on T2 (s), locomotor activity on T2 (number of crossed lines). The intraperitoneal route of administration were used to evaluate the promnesiant effects. Vehicle, or the compounds of formula I were administered 40 min before T1. Scopolamine was administered 30 min before T1.

Compounds of formula (I) according to the invention, tested according to the above protocol, displayed typically an activity of about 100 μmol/kg or less.

### Example 6

### Y-Maze Test

A non transgenic model of amyloid-induced memory deficit is used comprising: a bolus intracerebral injection of the aggregated β25-35 amyloid peptide into the lateral ventricle of mouse. Such injection induced 7-12 days later Congo-red stained amyloid-like deposits in the hippocampus and cortex. It also induced a variety of memory deficits observed in the spontaneous alternation, the inhibitory avoidance, or the Morris water maze task.

The spontaneous alternation in rat and mice refers to the spontaneous behavior of rodent to alternate in a Y or T-maze. Spontaneous alternation behavior has been ascribed to the operation of a variety of mechanism, but regardless of his ethological function, it is evident that the animal must remember which arm it had entered on a previous occasion to enable it to alternate its choice on a following trial. Therefore, spontaneous alternation has been embraced by behavioral pharmacologists as a quick and relatively simple test of memory devoid of fear, reward or re-enforcers.

A single unilateral intracerebral injection with 9 nmole aggregated β₂₅₋₃₅ amyloid peptide was administered in the right lateral ventricle according to the technique of Maurice et al. (Brain Research. 1996; 706:181-193).

The Y-maze was a three equal-size-arm maze (39 cm long) made of white PVC. The arms were oriented at 60 angles from each other. The Y-maze test was done 7-12 days post-amyloid administration under moderate lighting condition (200 lux), with moderate background music and mild eucalyptus odor. Compounds were given intraperitoneally 40 min before Y-maze trial.

Young Male Swiss mice began the single trial at the end of one arm, and were allowed to freely explore the Y-maze during 8 min. Number and sequence of arm visits was recorded. Alternation was defined as “a consecutive entry in three different arms”. The alternation percentage was computed with the following formula: “number of alternation” divided by “total number of arm visit” minus 2.

The test compound (+)-1-{[2-oxo-4-(3,4,5-trifluorophenyl)pyrrolidin-1-yl]methyl}-1H-imidazole-5-carbonitrile for instance displayed an activity at 8 and 32 μmol/kg.

1. A compound having an affinity to a SV2 protein and which counteracts the antiepileptic activity of levetiracetam for the treatment of a condition associated with enhancement or improvement of the cognitive ability or to counteract cognitive decline of a mammal.

2. The compound according to claim 1, having an affinity to the SV2A protein.

3. The compound according to claim 1, having an affinity to the SV2B protein.

4. The compound according to claim 1, having an affinity to the SV2C protein.

5. The compound according to claim 1, which has an affinity to a SV2 protein of at least pIC₅₀=5.

6. The compound according to claim 1, which diminishes the antiepileptic activity of levetiracetam by creating a shift of its ED₅₀ value against clonic seizures towards higher doses, at least by a factor of 2.

7. The compound according to claim 1, which is covered by formula (I) 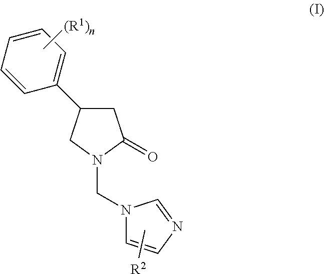
wherein
R¹ is an halogen atom, preferably a chlorine or a fluorine atom;
n is equal to 1, 2 or 3; and
R² is cyano.

8. The compound according to claim 1, which selected from the group consisting of:
(−)-1-{[2-oxo-4-(2,3,4-trifluorophenyl)pyrrolidin-1-yl]methyl}-1H-imidazole-5-carbonitrile
(+)-1-{[2-oxo-4-(2,3,4-trifluorophenyl)pyrrolidin-1-yl]methyl}-1H-imidazole-5-carbonitrile
(+)-1-{[2-oxo-4-(3,4,5-trifluorophenyl)pyrrolidin-1-yl]methyl}-1H-imidazole-4-carbonitrile
(+)-1-{[2-oxo-4-(2,4,5-trifluorophenyl)pyrrolidin-1-yl]methyl}-1H-imidazole-4-carbonitrile
(−)-1-{[2-oxo-4-(3,4,5-trifluorophenyl)pyrrolidin-1-yl]methyl}-1H-imidazole-5-carbonitrile
(+)-1-{[2-oxo-4-(3,4,5-trifluorophenyl)pyrrolidin-1-yl]methyl}-1H-imidazole-5-carbonitrile.

9. The compound according to claim 1, wherein said improvement of the cognitive ability comprises a disorder related to autism, dyslexia, attention deficit hyperactivity disorder, schizophrenia, obsessive compulsive disorders, psychosis, bipolar disorders, depression, Tourette's syndrome and disorders of learning in children, adolescents and adults, Age Associated Memory Impairment, Age Associated Cognitive Decline, Parkinson's Disease, Down's Syndrome, traumatic brain injury Huntington's Disease, Progressive Supranuclear Palsy (PSP), HIV, stroke, vascular diseases, Pick's or Creutzfeldt-Jacob diseases, multiple sclerosis (MS), other white matter disorders and drug-induced cognitive worsening.

10. A pharmaceutical composition containing a compound according to claim 1, as well as a suitable pharmaceutically acceptable excipient.

11. A method for the identification of compounds according to claim 1, said method comprising the steps of:
determining whether a test compound has a SV2 binding affinity and
determining whether a test compound counteracts the antiepileptic or anti-convulsive activity of levetiracetam.
